(19) United States
(12) Patent Application Publication
(10) Pub. No.: US 2019/0134128 A1
(43) Pub. Date: May 9, 2019

(54) METHOD FOR PRODUCING CELASTROL AND PENTACYCLIC TRITERPENE DERIVATIVES

(71) Applicant: PIERRE FABRE DERMO-COSMETIQUE, Boulogne-Billancourt (FR)

(72) Inventors: Thien NGUYEN, Roufflac-Tolosan (FR); Adrien COUSY, Beaumont Sur Leze (FR); Nicolas STEWARD, Pins-Justaret (FR)

(73) Assignee: PIERRE FABRE DERMO-COSMETIQUE, Boulogne-Billancourt (FR)

(21) Appl. No.: 16/099,952

(22) PCT Filed: May 12, 2017
(86) PCT No.: PCT/EP2017/061504

§ 371 Date: Nov. 8, 2018

(30) Foreign Application Priority Data
May 12, 2016 (FR) 1654240

(51) Int. Cl. A61K 36/37; A61P 29/00; A61P 31/04

(52) U.S. Cl. CPC A61K 36/37; A61K 2236/11; A61P 31/04; A61P 29/00

(57) ABSTRACT

The present invention relates to a method for producing a crude extract enriched in pentacyclic triterpenes, including celastrol, from cells of a plant from the Celastraceae family; an enriched crude extract that can be obtained using such a method; therapeutic applications of such an extract; and pharmaceutical and dermocosmetic compositions containing such an extract.

The present invention concerns a process for producing a crude extract enriched in pentacyclic triterpenes, notably celastrol, from a suspended plant cell culture (PCC) of a plant of the Celastraceae family.

Tripterygium wilfordii is a medicinal plant of the Celastraceae family which has been used for centuries in Southeast Asia (including China) to treat inflammatory disorders, autoimmune diseases and, more recently, cancer. Terpenes are among the most active components of the plant and are located mainly in the plant roots. Among these, a norfriedelane-type pentacyclic triterpene, celastrol, is being evaluated for its efficacy in the treatment, of obesity, rheumatoid arthritis, Crohn's disease and prostate cancer. Celastrol supply demands are therefore steadily increasing.

Inflammatory dermatosis, notably psoriasis or atopic dermatitis, has multifactorial origins or causes. The reasons for these so-called autoimmune diseases are not yet fully explained but are believed to be linked to immune system dysregulation, particularly in terms of cellular responses. Cellular responses can be classified into 3 categories: Th1, Th2 and, recently, Th17. Although psoriasis and atopic dermatitis have differences in clinical signs, they are believed to have similarities, particularly in terms of IL17 and IL22 expression characteristic of the Th17 response (Miyagaki et at 2015). IL17 and IL22 would therefore be attractive targets for treating these diseases. Thus, monoclonal antibodies against IL17, IL17R, IL22, IL23, TNF-alpha have been developed to block the Th17 pathway (Lowes et al. 2014). These molecules are effective for treating psoriasis but have many side effects and result in an exorbitant treatment cost.

Furthermore, it is also known that the Th17 pathway is strongly activated in lesions of patients with acne (Kelhala et al. 2014).

Celastrol is commonly obtained by solvent-based plant extraction processes from plants of the Celastraceae family. These biosynthesis methods involve cycles of eight to ten years for a young plant shoot to reach the maturity necessary to be sacrificed. Furthermore, the use of plant protection products makes the process costly: external contaminants (heavy metals) can accumulate and the crude extract produced contains a number of metabolites that require this plant extract to undergo several purification steps to isolate the triterpenic fraction. Furthermore, these processes have low yields of celastrol.

Total chemical synthesis of celastrol is possible (Camelio et al. 2015) but requires twenty or so steps, which substantially increases the final cost of obtaining the product.

An alternative solution has been to obtain celastrol from suspended cell cultures from stem cells generated from the plant roots or leaves. Recently, the Coppede team (Coppede et al. 2014) was able to obtain from an M. ilicifolia cell culture a larger amount of celastrol than that obtained by conventional extraction from the plant (0.304 mg celastrol per g dry weight, maximum concentration obtained after 8 days of culture, i.e. 8.85 times better than by the conventional extraction method). The team of Liu et al. (Liu et al. 2016) also showed that by adding MeJa (methyl jasmonate) (of the order of 50 μM) to a T. wilfordii plant cell culture, the amount of celastrol is increased by 4.82 times relative to a culture without MeJa: the concentration in mg celastrol per g dry weight is 0.752 mg/g (culture with MeJa) versus 0.154 mg/g (culture without MeJa).

These processes allow improved celastrol yields. However, there is still a genuine need for economically viable processes for obtaining large amounts of celastrol quickly.

The present invention provides a process for producing a crude extract enriched in celastrol from plant cells of a plant of the Celastraceae family.

Indeed, the inventors have developed a process for increasing, in a surprising manner, the celastrol yield in plant cell cultures from a T. wilfordii line by using and comparing various combinations of elicitors. In particular, the inventors observed that cell division and celastrol production are not concomitant. Surprisingly, they are even incompatible. To solve this problem, the inventors developed a process that includes a step of cell proliferation and a step of elicitation with a cocktail of eliciting agents that stops cell division. The inventors also demonstrated that the use of pairs of elicitors, notably methyl jasmonate (MeJa) and chitin, makes it possible to obtain a concentration of 16.6 mg/g celastrol per dry weight at the end of culture. The celastrol yield of this elicited culture is thus 22 times higher than that obtained by Liu et al. (op. cit.). Furthermore, the yield by the process according to the invention is 57 times higher than that obtained by Coppede et al. (op. cit.) without elicitation. The process according to the invention also makes it possible to enrich in celastrol derivatives, notably of the pentacyclic triterpene type, such as tingenin A (also called tingenone or maytenin), tingenin B (also called 22beta-hydroxy-tingenone), pristimerin and tripterygone.

Furthermore, the inventors have shown, surprisingly, that a crude extract enriched in pentacyclic triterpenes obtained by this process inhibits, with a dose effect, the Th17-specific interleukins induced in human CD4+ T cells.

The present invention thus concerns a process for producing a crude extract enriched in pentacyclic triterpenes comprising the following steps:

(i) a phase of proliferation of cells of a plant of the Celastraceae family in a proliferation medium,

(ii) a phase of elicitation by adding an elicitation cocktail to the cell culture obtained in step (I), said elicitation cocktail comprising at least one monocarboxylic compound-type elicitor and at least one biotic elicitor, and

(iii) preparation of a crude extract enriched in pentacyclic triterpenes from the cell culture obtained in step (ii).

Furthermore, the present invention concerns an extract obtainable by the process according to the invention, as well as uses thereof.

According to the invention, “pentacyclic triterpenes” means the pentacyclic triterpenes naturally produced by the cells of a plant of the Celastraceae family and notably celastrol (of formula 1), tingenin A (also called tingenone or maytenin) (of formula II), tingenin B (also called 22beta-hydroxy-tingenone) (of formula III), pristimerin (of formula IV) and tripterygone (of formula V), notably celastrol, tingenin A and tingenin B, notably celastrol.

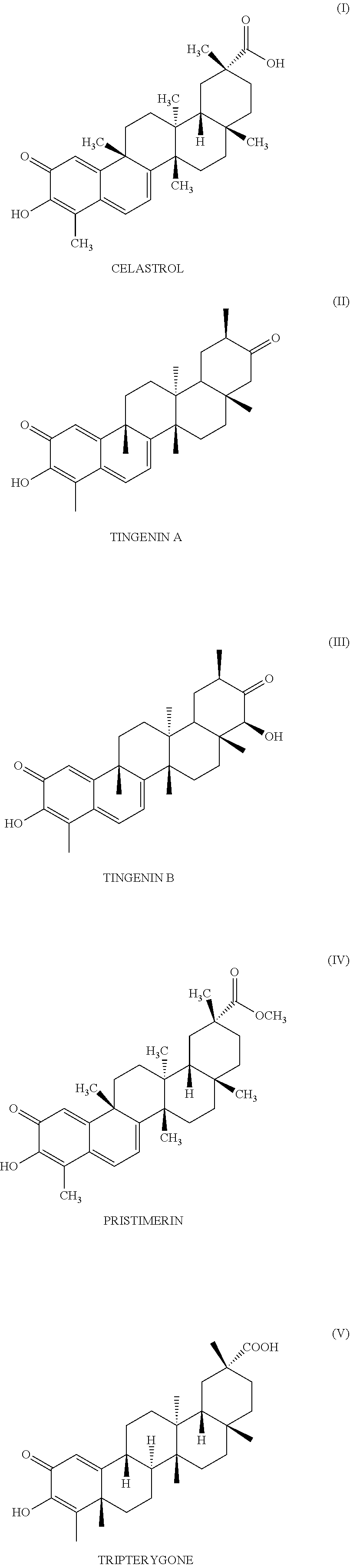

According to the invention, “crude extract enriched in pentacyclic triterpenes” means an enriched crude extract comprising an amount of pentacyclic triterpenes at least 2 times, notably at least 5 times, notably at least 10 times, notably between 10 and 100 times higher than that obtained by a process in the absence of an elicitation phase according to the invention.

According to the invention, “crude extract enriched in celastrol” means an enriched crude extract comprising an amount of celastrol at least 2 times, notably at least 5 times, notably at least 10 times, notably between 10 and 60 times, notably between 10 and 20 times higher than that obtained by a process in the absence of an elicitation phase according to the invention.

According to the invention, “plant of the Celastraceae family” means all plants belonging to this family, and notably plants of the genera Tripterygium, Bhesa, Kokkona, Catha, Euonymus, Orthosphenia, Dicarpellum, Celastrus, Maytenus, Peritassa and Rzedowskia, notably Tripterygium and Celastrus. Among the genus Tripterygium, particular mention may be made of the species Tripterygium wilfordii, Tripterygium regelii, Tripterygium hypoglaucum and Tripterygium articulate, notably Tripterygium wilfordii.

According to the invention, “cells of a plant of the Celastraceae family” means the cells of any part of the plant: seeds, roots, aerial parts, and notably aerial parts.

According to the invention, “aerial parts” means the parts of the plant located above the ground, for example leaves, stems, petioles and/or inflorescences, notably leaves.

According to the invention, “phase of proliferation of cells of a plant of the Celastraceae family” means a phase in which the cells of a plant of the Celastraceae family are suspended in a proliferation medium and under conditions suited to their proliferation. These cells may notably be obtained from calluses before being suspended. If necessary, the cell suspensions may be regularly reseeded to maintain them under proliferation conditions.

According to the invention, “callus” means a cluster of dedifferentiated cells, also called stem cells or meristematic cells.

Callus induction may be achieved by any method known to the skilled person. Calluses according to the invention may notably be obtained in the manner described below.

The induction of calluses from a tissue explant from a part, notably an aerial part, of a plant of the Celastraceae family, for example Tripterygium wilfordii, is well known to the skilled person.

The induction of calluses may notably be carried out by:

obtaining a tissue explant from the plant, for example a piece of leaf about 1 cm² in size,

culturing the explant on an agar proliferation medium according to the invention (for example by adding 4 to 12 g/L agar, for example about 8 g/L agar, to the proliferation medium according to the invention),

incubating, notably in the dark, at a temperature of about 25-30° C., for example at about 27° C. to 28° C.

Step (i): Cell Proliferation Phase

The skilled person familiar with cultures of cells of a plant of the Celastraceae family will easily be able to determine the composition of the proliferation medium necessary for their proliferation. Preferentially, this proliferation medium will allow the proliferation of cells in dedifferentiated, i.e. totipotent, forms. Maintenance in dedifferentiated form may notably be achieved by using specific cytokinin/auxin ratios in the proliferation medium.

The “proliferation medium” according to the invention may notably comprise:

at least one macroelement, notably selected from NH₄NO₃, KNO₃, CaCl₂.2H₂O, MgSO₄.7H₂O, KH₂PO₄ and a mixture thereof, for example at a total macroelement concentration comprised between 1000 and 9000 mg/L, for example between 3000 and 8000 mg/L of proliferation medium: during the proliferation phase, the total macroelement concentration of the proliferation medium will notably be comprised between 3000 and 5500 mg/L of proliferation medium;

at least one microelement, notably selected from KI, H₃BO₃, MnSO₄.4H₂O, ZnSO₄.H₂O, Na₂MoO₄.2H₂O, CuSO₄.5H₂O, CoCl₂.6H₂O, FeSO₄.7H₂O, Na₂EDTA.2H₂O and a mixture thereof, for example at a total microelement concentration comprised between 10 and 200 mg/L, notably between 50 and 150 mg/L of proliferation medium;

at least one vitamin, notably selected from myo-inositol, nicotinic acid, pyridoxine-HCl, thiamine-HCl and a mixture thereof, for example at a total vitamin concentration ranging from 0.01 to 3 g/L, notably from 0.05 to 1 g/L of proliferation medium;

at least one amino acid, notably glycine, for example at a total amino acid concentration ranging from 0.15 to 5 mg/L, notably between 1 and 4 mg/L of proliferation medium: during the proliferation phase, the amino acid concentration of the proliferation medium will notably be comprised between 1 and 2.5 mg/L of proliferation medium;

at least one carbon source, notably sucrose, for example at a total carbon source concentration of 10 to 70 g/L of proliferation medium, for example at about 30 g/L;

at least one plant hormone (also called plant growth hormone or plant growth factor or plant growth regulator) notably selected from one or more cytokinins, notably kinetin and/or 6-furfurylaminopurine, one or more auxins, notably 2,4-dichlorophenoxyacetic acid (2,4-D) and/or naphthalene acetic acid (NAA), and a mixture thereof. During the proliferation phase, the proliferation medium will notably comprise at least one cytokinin and at least one auxin.

The plant growth hormones will notably be added to the proliferation medium at a concentration and ratio allowing the proliferation of cells in dedifferentiated forms. They will notably be selected from kinetin, 6-furfurylaminopurine, 2,4-dichlorophenoxyacetic acid (2,4-D), naphthalene acetic acid (NAA) and a mixture thereof; notably selected from kinetin, 2,4-dichlorophenoxyacetic acid (2,4-D), naphthalene acetic acid (NAA) and a mixture thereof. In particular, it may be a mixture of kinetin, 2,4-dichlorophenoxyacetic acid (2,4-D) and naphthalene acetic acid.

The concentration of auxins according to the invention will notably be comprised between 0.001 and 10 mg/L of proliferation medium, for example between 0.1 and 3 mg/L of proliferation medium.

The concentration of cytokinins according to the invention will notably be between 0.01 and 0.5 mg/L of proliferation medium, notably between 0.05 and 0.15 mg/L.

In one embodiment, the auxin/cytokinin hormone ratio will be comprised between 0.2 to 2.5/0.01 to 0.5, notably between 1 to 2/0.05 to 0.2 and notably will be around 1.5/0.1.

Notably, the proliferation medium according to the invention may comprise 1.5 mg/L of auxin, notably 2,4-dichlorophenoxyacetic acid (2,4-D) and naphthalene acetic acid (NAA), and 0.1 mg/L of cytokinin, notably kinetin.

The proliferation medium will be sterile and preferentially at a pH close to neutral.

An exemplary proliferation medium suited to the proliferation of cells of a plant of the Celastraceae family according to the invention is notably described by Murashige & Skoog (1962) or in the examples in the present application.

This proliferation medium may for example have the following composition (concentrations are expressed in relation to the volume of cell-free proliferation medium): Macroelements: NH₄NO₃ at 1650 mg/L, KNO₃ at 1900 mg/L, CaCl₂.2H₂O at 440 mg/L, MgSO₄.7H₂O at 370 mg/L, KH₂PO₄ at 170 mg/L; Microelements: KI at 0.83 mg/L, H₃BO₃ at 6.2 mg/L, MnSO₄.4H₂O at 22.3 mg/L, ZnSO₄.H₂O at 6.6 mg/L, Na₂MoO₄.2H₂O at 0.25 mg/L, CuSO₄.5H₂O at 0.025 mg/L, CoCl₂.6H₂O at 0.025 mg/L, FeSO₄.7H₂O at 27.8 mg/L, Na₂EDTA.2H₂O at 37.3 mg/L; Vitamins: myo-inositol at 100 mg/L, nicotinic acid at 0.5 mg/L, pyridoxine-HCl at 0.5 mg/L, thiamine-HCl at 0.5 mg/L; Amino acids: glycine at 2 mg/L; Carbon source: sucrose at 30 g/L; and Plant hormones: naphthalene acetic acid at 1 mg/L, 2,4-dichlorophenoxyacetic acid at 0.5 mg/L, kinetin at 0.1 mg/L, all adjusted to pH 6 before sterilization (for example by autoclaving for 20 min at 121° C. or by filtration through a 0.2 μm filter).

Alternatively, the proliferation medium may have the following composition: (concentrations are expressed in relation to the volume of cell-free proliferation medium): Macroelements: NH₄NO₃ at 1650 mg/L, KNO₃ at 2500 mg/L, CaCl₂.2H₂O at 440 mg/L, MgSO₄.7H₂O at 370 mg/L, KH₂PO₄ at 130 mg/L; Microelements: KI at 0.41 mg/L, H₃BO₃ at 6.2 mg/L, MnSO₄.4H₂O at 22.3 mg/L, ZnSO₄.H₂O at 7.5 mg/L, Na₂MoO₄.2H₂O at 0.25 mg/L, CuSO₄.5H₂O at 0.025 mg/L, CoCl₂.6H₂O at 0.025 mg/L, FeSO₄.7H₂O at 19.85 mg/L, Na₂EDTA.2H₂O at 26.64 mg/L; Vitamins: myo-inositol at 50 mg/L, nicotinic acid at 0.25 mg/L, pyridoxine-HCl at 0.25 mg/L, thiamine-HCl at 0.25 mg/L; Carbon source: sucrose at 30 g/L; and Plant hormones: kinetin at 0.083 mg/L, 2,4-dichlorophenoxyacetic acid (2,4-D) at 0.575 mg/L, naphthalene acetic acid (NAA) at 0.350 mg/L.

Alternatively, the proliferation medium according to the invention may have the following composition (concentrations are expressed in relation to the volume of cell-free proliferation medium): NH₄NO₃ at 20 mM, KNO₃ at 19 mM, CaCl₂.2H₂O at 3 mM, MgSO₄.7H₂O at 1.50 mM, KH₂PO₄ at 1.2 mM, KI at 0.005 mM, H₃BO₃ at 0.1 mM, MnSO₄.4H₂O at 0.1 mM, ZnSO₄.H₂O at 0.04 mM, Na₂MoO₄.2H₂O at 0.001 mM CuSO₄.5H₂O at 0.0001 mM, COCl₂.6H₂O at 0.0001 mM, FeSO₄.7H₂O at 0.1 mM, Na₂EDTA.2H₂O at 0.1 mM, myo-inositol 0.5 mM, nicotinic acid at 0.004 mM, pyridoxine-HCl at 0.002 mM, thiamine-HCl at 0.0015 mM, glycine at 0.03 mM, sucrose at 87.6 mM, naphthalene acetic acid at 0.005 mM, 2,4-dichlorophenoxyacetic acid at 0.002 mM, and kinetin at 0.0005 mM.

Seeding of the proliferation medium may be carried out by suspending callus cells at a concentration comprised between 20 and 300 g in 1 L of proliferation medium and preferably between 100 and 200 g in 1 L of proliferation medium, for example about 150 g in 1 L of proliferation medium.

The proliferation phase will take place under biomass multiplication conditions. According to the invention, “biomass multiplication conditions” means notably the temperature, duration, shaking, and light conditions necessary for the proliferation of suspended cells. The skilled person familiar with cultures of cells of a plant of the Celastraceae family will be able to easily determine the biomass multiplication conditions. In one embodiment according to the invention, the biomass proliferation step will take place in the dark, at a temperature comprised between 20 and 35° C., notably between 27 and 28° C., notably at about 27 or 28° C., notably with shaking between 100 and 200 rpm, notably at about 125 rpm (22.5 mm orbital) and for a duration comprised between 10 and 30 days, notably 15 days of culture.

During this step, the cells can be “subcultured” or propagated, for example every 7 to 15 days. The subculturing of cells is well known by the skilled person: it may notably consist in diluting part of the cell culture in concentrated fresh medium. For example, ⅕^{th} of the culture is resuspended in a volume of fresh medium corresponding to the volume of the initial culture. It makes is possible to maintain the cell line in liquid medium in a state of proliferation.

Step (ii)—Elicitation Phase

After the proliferation phase, the cells obtained in phase (i) are elicited by adding an elicitation cocktail. The proliferation medium to which the elicitation cocktail has been added will be called “elicitation medium” according to the present invention.

The elicitation phase is the phase in which the cells are maintained in a physiological state promoting the biosynthesis of secondary metabolites such as pentacyclic triterpenes.

The production of pentacyclic triterpenes, notably celastrol, occurs during the elicitation phase in the cytosol of the cell and may partially diffuse into the elicitation medium. The elicitation phase within the meaning of the present invention therefore corresponds to the phase of production (biosynthesis) of celastrol and derivatives thereof (pentacyclic triterpenes).

Elicitation will preferentially be carried out after a proliferation phase of 7 to 21 days, notably 12 to 20 days, notably 15, 16 or 17 days (notably without subculturing). Alternatively, the elicitation cocktail can be added when the cell concentration obtained during the proliferation phase is double, notably more than double, the initial cell concentration in the proliferation medium. The elicitation cocktail may notably be added when the cell concentration is greater than 200 g/L, for example between 200 and 400 g/L, notably about 300 g/L (by weight of cells per litre of proliferation medium). In one embodiment, the elicitation cocktail will be added to a culture whose cell concentration has increased from 150 to 200 g/L at the beginning of the proliferation phase to a concentration comprised between 300 and 400 g/L at the end of the proliferation phase.

Following the proliferation phase, the plant cells have consumed most or all of the elements contained in the proliferation medium, and in particular the carbon sources such as sucrose. It may therefore be necessary to restore the composition of the medium before or at the same time the elicitation cocktail is added.

To restore the composition of the medium, the cell culture obtained after the proliferation step may notably be concentrated, for example by settling or filtration, then fresh proliferation medium is added to the cells thus obtained. In this case, the cells will be resuspended in the fresh proliferation medium so as to obtain a concentration comprised between 200 g/L and 400 g/L, for example between 250 and 350 g/L, notably about 300 g/L (by weight of cells per litre of proliferation medium).

Alternatively, a concentrated mixture can be added to the cell culture to restore the concentrations of the elements in the proliferation medium. The concentrated mixture will notably be added just before, just after or at the same time as the elicitation cocktail. For example, ⅕^{th} of the cell culture may be replaced by an equivalent volume of proliferation medium concentrated 5 times.

The concentration of elements in the proliferation medium is considered to be close or equivalent to zero after 14, 15 or 16 days of proliferation phase. In particular, the concentration of mineral elements (more particularly the macroelements and microelements) and carbonaceous elements (more particularly the carbon sources) is considered to be close or equivalent to zero after 14, 15 or 16 days of proliferation phase.

In one embodiment, the proliferation medium according to the invention at the beginning of the elicitation phase will comprise, among other things:

at least one macroelement, notably selected from NH₄NO₃, KNO₃, CaCl₂.2H₂O, MgSO₄.7H₂O, KH₂PO₄ and a mixture thereof, for example at a total macroelement concentration comprised between 5000 and 8000 mg/L, preferentially greater than 6000 mg/L of proliferation medium, advantageously between 6000 and 8000 mg/L;

at least one microelement, notably selected from KI, H₃BO₃, MnSO₄.4H₂O, ZnSO₄.H₂O, Na₂MoO₄.2H₂O, CuSO₄.5H₂O, CoCl₂.6H₂O, FeSO₄.7H₂O, Na₂EDTA.2H₂O and a mixture thereof, for example at a total microelement concentration comprised between 10 and 200 mg/L, notably between 50 and 150 mg/L of proliferation medium;

at least one vitamin, notably selected from myo-inositol, nicotinic acid, pyridoxine-HCl, thiamine-HCl and a mixture thereof, for example at a total vitamin concentration ranging from 0.01 to 3 g/L, notably from 0.05 to 1 g/L of proliferation medium;

at least one amino acid, notably glycine, for example at a concentration in the proliferation medium comprised between 3 and 4 mg/L of proliferation medium;

at least one carbon source, notably sucrose, for example at a total carbon source concentration of 10 to 70 g/L of proliferation medium, for example at about 30 g/L;

In one embodiment the proliferation medium at the beginning of the elicitation phase will not comprise, or will comprise a negligible amount, notably less than 0.001 g/ml, of cytokinin and of auxin.

The proliferation medium during the elicitation phase will be advantageously sterile and preferentially at a pH close to neutral.

The proliferation medium during the elicitation phase may notably have the following composition: Macroelements: NH₄NO₃ at 2.8 g/L, KNO₃ at 3 g/L, CaCl₂.2H₂O at 0.45 g/L, MgSO₄.7H₂O at 74 mg/L, KH₂PO₄ at 34 mg/L; Microelements: KI at 0.16 mg/L, H₃BO₃ at 6.2 mg/L, MnSO₄.4H₂O at 18.5 mg/L, ZnSO₄.H₂O at 6.6 mg/L, Na₂MoO₄.2H₂O at 0.25 mg/L, CuSO₄.5H₂O at 0.025 mg/L, CoCl₂.6H₂O at 0.025 mg/L, FeSO₄.7H₂O at 28 mg/L, Na₂EDTA.2H₂O at 37 mg/L; Vitamins: myo-inositol at 250 mg/L, nicotinic acid at 1.7 mg/L, pyridoxine-HCl at 1 mg/L, thiamine-HCl at 1 mg/L, Amino acid: glycine at 4 mg/L; Carbon source: sucrose at 30 g/L.

According to the invention, “elicitation cocktail” means a cocktail that stops cell division. This elicitation cocktail comprises at least one monocarboxylic compound-type elicitor and at least one biotic elicitor. The elicitation cocktail is introduced into the culture medium using, for example, concentrated stock solutions.

According to the invention, “monocarboxylic compound-type elicitor” means more particularly an elicitor selected from the group comprising, preferably consisting of, 5-chlorosalicylic acid (5-chloroSA), salicylic acid (SA), acetylsalicylic acid (ASA), a methyl ester, notably methyl jasmonate (MeJa), and a mixture thereof. In one embodiment according to the invention, the monocarboxylic compound-type elicitor is methyl jasmonate, salicylic acid and/or 5-chlorosalicylic acid, notably methyl jasmonate. The monocarboxylic compound-type elicitor will notably be added so as to obtain a final concentration comprised between 0.005 and 0.1 g/L, notably 0.01 and 0.05 g/L of elicitation medium, notably 0.002 and 0.004 g/L of elicitation medium.

According to the invention, “biotic elicitor” means more particularly a biotic elicitor selected from the group comprising, preferably consisting of, an N-acetylaminoglucosamine, notably chitin, chitosan, extracts of microorganisms or fungi, oligosaccharides (polysaccharides, pectins, cellulose). In one embodiment, the biotic elicitor is chitin. Chitin is a linear polymer with the repeating unit beta-1,4-N-acetyl-D-glucosamine. The biotic elicitor will notably be added so as to obtain a final concentration comprised between 0.05 and 50 g/L of elicitation medium, for example from 0.1 to 10 g/L, for example from 0.5 to 7 g/L, notably from 1 to 5 g/L of elicitation medium.

In one embodiment, the elicitation cocktail comprises methyl jasmonate, notably at a final concentration in the elicitation medium between 0.002 and 0.005 g/L, and chitin, notably at a final concentration between 1 and 4 g/L of elicitation medium.

In one embodiment, the elicitation cocktail according to the invention will further comprise at least one cell differentiation factor for plant cells and/or at least one precursor of the terpene synthesis pathway.

The “cell differentiation factor for plant cells” according to the invention may notably be selected from the group comprising, preferably consisting of, a cytokinin, notably benzylaminopurine (BAP), abscisic acid, kinetin, thidiazuron, 6-γ,γ-dimethylallylaminopurine (2iP or isopentenyladenine) or zeatin, a gibberellin and a mixture thereof, notably BAP and/or 2iP, in particular 2iP.

The “precursor of terpene synthesis” according to the invention may notably be selected from the group comprising, preferably consisting of, sodium pyruvate; potassium, pyrophosphate; mevalonic acid; geraniol; farnesol; isopentenyl, dimethylallyl, including the pyrophosphate forms thereof; sodium acetate; pyruvic acid and mixtures thereof, notably geraniol, farnesol, sodium pyruvate, potassium pyrophosphate and mixtures thereof, such as sodium pyruvate and/or potassium pyrophosphate.

BAP may notably be used at a final concentration in the elicitation medium comprised between 0.01 and 5 mg/L, for example between 0.5 and 5 mg/L of elicitation medium.

5-Chlorosalicylic acid (5-chloroSA) may notably be used at a final concentration in the elicitation medium comprised between 0.1 and 15 mg/L.

Salicylic acid may notably be used at a final concentration in the elicitation medium comprised between 0.1 and 100 mg/L, for example between 20 and 60 mg/L, for example about 45 mg/L.

Farnesol may notably be used at a final concentration in the elicitation medium of 1 to 100 mg/L, for example 15 to 30 mg/L, for example at about 30 mg/L.

Geraniol may notably be used at a final concentration in the elicitation medium of 1 to 100 mg/L, for example 20 to 30 mg/L.

Sodium pyruvate may notably be used at a final concentration in the elicitation medium of 100 to 5000 mg/L, for example 500 to 2000 mg/L.

Potassium pyrophosphate may notably be used at a final concentration in the elicitation medium of 1 to 2000 mg/L, for example 100 to 1000 mg/L of elicitation medium.

2iP may notably be used at a final concentration in the elicitation medium of 0.005 to 10mg/L, for example 0.01 mg/L to 3 mg/L, for example 0.1 to 2 mg/L.

In one embodiment according to the invention, the elicitation cocktail comprises methyl jasmonate, chitin, sodium pyruvate, potassium pyrophosphate or a mixture thereof.

In one embodiment according to the invention, the elicitation cocktail comprises methyl jasmonate, chitin, sodium pyruvate, potassium pyrophosphate, and optionally BAP and/or 2iP.

In one embodiment according to the invention, the elicitation cocktail comprises or consists of (the concentrations given between parentheses correspond to the concentration in the elicitation medium, the initial cocktail which may be more or less concentrated as a function of the intended dilution) sodium pyruvate (500 to 2000 mg/L), potassium pyrophosphate (100 to 1000 mg/L), 2iP (0.1 to 2 mg/L), methyl jasmonate (0.002 to 0.005 g/L) and chitin (1 to 4 g/L).

In another embodiment, the elicitation cocktail comprises or consists of (the concentrations given between parentheses correspond to the concentration in the elicitation medium, the initial cocktail which may be more or less concentrated as a function of the intended dilution) benzylaminopurine (BAP) (0.5 to 5 mg/L, notably 0.5 to 3 mg/L); 5-chlorosalicylic acid (5-chloroSA) (2 to 6 mg/L, notably 3 to 5 mg/L, for example at about 3 or at about 5 mg/L), acetylsalicylic acid (ASA) and/or salicylic acid (SA) (20 to 60 mg/L, notably 30 to 50 mg/L, notably 33 to 45 mg/L); methyl jasmonate (Meta) (0.002 to 0.005 g/L, notably 10 to 40 mg/L); chitin (1 to 4 g/L); and farnesol (F—OH) (19 to 40 mg/L) and/or geraniol (20 to 30 mg/L).

In another embodiment the elicitation cocktail comprises or consists of (the concentrations given between parentheses correspond to the concentration in the elicitation medium, the initial cocktail which may be more or less concentrated as a function of the intended dilution) sodium pyruvate (500 to 2000 mg/L), potassium pyrophosphate (100 to 1000 mg/L), 2iP (0.1 to 1 mg/L), methyl jasmonate (MeJa) (0.002 to 0.005 g/L, notably 10 to 40 mg/L) and chitin (1 to 4 g/L).

In another embodiment, the elicitation cocktail comprises or consists of (the concentrations given between parentheses correspond to the concentration in the elicitation medium, the cocktail may be more or less concentrated as a function of the intended dilution) sodium pyruvate (about 1.5 g/L), potassium pyrophosphate (about 0.4 g/L), 2iP (about 0.4 mg/L), methyl jasmonate (MeJa) (about 0.03 mg/L), chitin (about 2 g/L).

During the elicitation phase, after addition of the elicitation cocktail, the culture is maintained under shaking, notably between 50 and 200 rpm, notably at about 125 rpm, for a period comprised between 3 and 30 days, notably between 10 and 25 days, notably between 12 and 15 days, in particular at a temperature comprised between 20 and 35° C., notably at about 27° C. and advantageously with a dissolved oxygen level in the culture medium of 2 to 40%, preferably at about 16%. A supply of sterile air sufficiently enriched in oxygen may be provided if necessary, notably in the headspace of the bioreactor or by diffusion in the medium. Preferably, the elicitation phase (iii) is conducted in the dark. During the elicitation phase, the cell culture is preferentially not subcultured.

Step ((iii)—Phase of Preparation of the Crude Extract Enriched in Pentacyclic Triterpenes

After the elicitation phase, the process according to the invention comprises a step of preparation of a crude extract enriched in pentacyclic triterpenes, notably celastrol.

The enriched crude extract may notably be obtained by separation of the biomass and the culture supernatant. Separation may notably be carried out by direct filtration (0-50 μm), by centrifugation or by cell settling.

In one embodiment, the enriched crude extract according to the invention may consist of the culture supernatant thus recovered.

The enriched crude extract according to the invention may also be obtained after biomass lysis. For example, the cells contained in the recovered biomass may be lysed by a physical (sonication or grinding) or chemical (acid lysis) method then this lysate will be subjected to solvent extraction. The organic phase, notably comprising triterpenes from the cytosol, is then recovered, notably by settling or centrifugation. The solvent will notably be an ester-type solvent, and more particularly an alkyl acetate, the alkyl being more particularly linear or branched with 1 to 6 carbon atoms, notably ethyl acetate or isopropyl acetate. The volume of solvent used will be 2 volumes per weight of biomass.

Preferentially, the enriched crude extract according to the invention will correspond to the organic phase thus recovered.

Alternatively, the enriched crude extract according to the invention may be obtained after the solvent is evaporated and replaced notably by a carrier suited to the field of use of the extract (cosmetics, pharmaceuticals) and notably vegetable oils.

The enriched crude extract according to the invention is not purified. The enriched crude extract may optionally be purified in a subsequent step.

When the enriched crude extract is purified, to give a purified extract, the process according to the invention further comprises a step (iv) of obtaining a purified extract of one or more pentacyclic triterpenes from the enriched crude extract obtained in step (iii).

The purified extract according to the invention will then comprise more than 90%, notably more than 95%, notably more than 98% of one or more pentacyclic triterpenes according to the invention. In one embodiment, the purified extract according to the invention comprises more than 90%, notably more than 95%, notably more than 98% celastrol, notably 100% celastrol.

Purification of the enriched extract according to the invention may notably be carried out by a phase of separation of the pentacyclic triterpene(s), notably celastrol, tingenin A and tingenin B, notably by HPLC (high-performance liquid chromatography) fractionation, in particular the peaks at 426 nm comprising celastrol, tingenin A and tingenin B elute at 10.5 min, 8.2 min and 6.5 min, respectively. Purification of celastrol may notably be carried out as described in the examples in the present application.

The present invention further relates to an enriched crude extract obtainable by the process according to the invention.

Another object of the invention concerns a pharmaceutical, such as dermatological, or dermocosmetic composition comprising an enriched crude extract obtainable by the process according to the invention and one or more pharmaceutically or dermocosmetically acceptable excipients.

The pharmaceutically or dermocosmetically acceptable excipients may be any excipient among those known to the person skilled in the art. The composition according to the invention will notably be a topical composition, notably in the form of a cream, a lotion, a gel, an ointment, an emulsion, a microemulsion, a spray, etc.

The pharmaceutical, such as dermatological, or dermocosmetic composition according to the invention may in particular contain additives and formulation aids, such as emulsifiers, thickeners, gelling agents, water fixatives, spreading agents, stabilizers, dyes, fragrances and preservatives.

Another object according to the invention is an enriched crude extract obtained by the process according to the invention, for use as a medicinal product, notably in the treatment or prevention of inflammatory dermatosis inducing a TH17-mediated cellular response.

The skilled person will be able to easily determine the inflammatory dermatoses that induce a TH17-mediated cellular response. The skilled person will notably be able to measure the expression level of IL-22, IL-17 and/or TNF-α of a lesioned area relative to a control sample.

The inflammatory dermatosis according to the invention may notably be selected from the group comprising, preferably consisting of, psoriasis, atopic dermatitis and acne.

The present invention is illustrated by the non-limiting figures and examples detailed below.

### FIGURES

FIG. 1: Curve of growth (solid line) of a Tripterygium wilfordii cell culture in g/L wet biomass (FW) as a function of time in days and of celastrol concentration (dotted line) in mg of celastrol per L cell suspension as a function of time in days.

FIG. 2: HPLC chromatogram (λ=426 nm) of the enriched crude extract of Tripterygium wilfordii obtained at day 33.

FIG. 3: Percentage induction of IL-17A synthesis by CD4+ T lymphocytes after incubation with samples 2, 3, 4 and 5 compared with the negative control incubated with sample 1 followed by stimulation with anti-CD3 and anti-CD28 antibodies (*** p-value<0.001).

FIG. 4: Percentage induction of INF-γ synthesis by CD4+ T lymphocytes after incubation with samples 2, 3, 4 and 5 compared with the negative control incubated with sample 1 followed by stimulation with anti-CD3 and anti-CD28 antibodies (*** p-value<0.001).

FIG. 5: Percentage induction of IL-22 synthesis by CD4+ T lymphocytes after incubation with samples 2, 3, 4 and 5 compared with the negative control incubated with sample 1 followed by stimulation with anti-CD3 and anti-CD28 antibodies (*** p-value<0.001).

FIG. 6: Percentage induction of TNF-α synthesis by CD4+ T lymphocytes after incubation with samples 2, 3, 4 and 5 compared with the negative control incubated with sample 1 followed by stimulation with anti-CD3 and anti-CD28 antibodies (*** p-value<0.001).

FIG. 7: Percentage induction of IL-6 synthesis by CD4+ T lymphocytes after incubation with samples 2, 3, 4 and 5 compared with the negative control incubated with sample 1 followed by stimulation with anti-CD3 and anti-CD28 antibodies (*** p-value<0.001).

### EXAMPLES

### Example 1

### Cell Dedifferentiation Protocol

Calluses are obtained from Tripterygium wilfordii leaf explants.

Explants are sterilized with 70% ethanol, then with sodium hypochlorite with 2.5% active chlorine, then rinsed with sterile demineralized water. Optionally, washing with 7% hydrogen peroxide before rinsing with sterile demineralized water is possible.

Leaves are cut into pieces, for example into squares of about 8-10 mm on a side. Leaf explants are deposited on agar proliferation medium.

The composition of the proliferation medium is as follows:

Macroelements: NH₄NO₃ at 1650 mg/L, KNO₃ at 1900 mg/L, CaCl₂.2H₂O at 440 mg/L, MgSO₄.7H₂O at 370 mg/L, KH₂PO₄ at 170 mg/L,

Microelements: KI at 0.83 mg/L, H₃BO₃ at 6.2 mg/L, MnSO₄.4H₂O at 22.3 mg/L, ZnSO₄.H₂O at 6.61 mg/L, Na₂MoO₄.2H₂O at 0.25 mg/L, CuSO₄.5H₂O at 0.025 mg/L, CoCl₂.6H₂O at 0.025 mg/L, FeSO₄.7H₂O at 27.8 mg/L, Na₂EDTA.2H₂O at 37.3 mg/L,

Vitamins: myo-inositol at 100 mg/L, nicotinic acid at 0.5 mg/L, pyridoxine-HCl at 0.5 mg/L, thiamine-HCl at 0.5 mg/L,

Amino acid: glycine at 2 g/L,

Carbon source: sucrose at 30 g/L,

Plant hormones: kinetin at 0.1 mg/L, 2,4-dichlorophenoxyacetic acid (2,4-D) at 0.5 mg/L, naphthalene acetic acid (NAA) at 1 mg/L.

The proliferation medium is gelled by adding agar at 8-12 g/L, its pH is adjusted to pH 6±0.5 (with KOH, 1M) before autoclaving for 20 min at 121° C. Petri dishes containing the explants are incubated in the dark at 27-28° C.

Calluses are subcultured every month on the same agar medium. To that end, the calluses obtained are detached from the leaf explant and deposited on fresh agar proliferation medium.

### Example 2

### Formulation of Culture and Propagation Media (Proliferation Phase)

After a few months of subculturing, friable calluses are obtained and transferred to liquid proliferation medium.

The proliferation medium has, for example, the composition indicated below:

Macroelements: NH₄NO₃ at 1650 mg/L, KNO₃ at 2500 mg/L, CaCl₂.2H₂O at 440 mg/L, MgSO₄.7H₂O at 370 mg/L, KH₂PO₄ at 130 mg/L,

Microelements: KI at 0.41 mg/L, H₃BO₃ at 6.2 mg/L, MnSO₄.4H₂O at 22.3 mg/L, ZnSO₄.H₂O at 7.5 mg/L, Na₂MoO₄.2H₂O at 0.25 mg/L, CuSO₄.5H₂O at 0.025 mg/L, CoCl₂.6H₂O at 0.025 mg/L, FeSO₄.7H₂O at 19.85 mg/L, Na₂EDTA.2H₂O at 26.64 mg/L,

Vitamins: myo-inositol at 50 mg/L, nicotinic acid at 0.25 mg/L, pyridoxine-HCl at 0.25 mg/L, thiamine-HCl at 0.25 mg/L,

Carbon sources: sucrose at 30 g/L

Plant hormones: kinetin at 0.083 mg/L, 2,4-dichlorophenoxyacetic acid (2,4-D) at 0.575 mg/L, naphthalene acetic acid (NAA) at 0.350 mg/L.

The pH of the medium is adjusted to pH 6±0.5 (by adding KOH, 1M) before an appropriate sterilization treatment, for example autoclaving at 121° C. for a minimum duration of 20 minutes or by 0.2 μm sterilizing filtration.

Cell suspension is achieved by depositing about 40 g of friable callus in a 200 ml Erlenmeyer flask containing the propagation medium, incubation for one week on a shaker table at 100 rpm (rotations per minute) in the dark at 27-28° C. The cell supernatant is collected by pipette, leaving the residual callus clusters. The cell suspension obtained is cultured for 15 days then propagated by dilution to ⅕^{th} in fresh medium every 15 days.

The Erlenmeyer flasks are filled to 40% (80 mL) and the inoculation rate by cell suspension transfer is 20-25% of the volume, or about 160 g/L of fresh biomass. The culture is then conducted for 15 days in the dark at 27-28° C. with orbital shaking at 110-120 rpm. At this stage the biomass is present at a concentration of about 300 g/L of fresh biomass per litre of suspension.

### Example 3

### Production of Triterpenes in an Erlenmeyer Flask (Phase of Elicitation and of Preparation of the Enriched Crude Extract)

Elicitation Phase:

After 15 days of culture, ⅕^{th} of the cell culture is removed from the Erlenmeyer flask and 20 mL of concentrated proliferation medium is added to the Erlenmeyer flask. The composition of the concentrated medium is as follows: Macroelements: NH₄NO₃ at 13.9 g/L, KNO₃ at 15.2 g/L, CaCl₂.2H₂O at 2.2 g/L, MgSO₄.7H₂O at 370 mg/L, KH₂PO₄ at 170 mg/L; Microelements: KI at 0.83 mg/L, H₃BO₃ at 31.2 mg/L, MnSO₄.4H₂O at 91.5 mg/L, ZnSO₄.H₂O at 33.05 mg/L, Na₂MoO₄.2H₂O at 1.25 mg/L, CuSO₄.5H₂O at 0.125 mg/L, CoCl₂.6H₂O at 0.125 mg/L, FeSO₄.7H₂O at 139 mg/L, Na₂EDTA.2H₂O at 186.5 mg/L; Vitamins: myo-inositol at 1250 mg/L, nicotinic acid at 8.5 mg/L, pyridoxine-HCl at 5 mg/L, thiamine-HCl at 5 mg/L; Amino acid: glycine at 20 mg/L; Carbon source: sucrose at 150 g/L (dissolved in demineralized water).

The elicitation cocktail is then added to the proliferation medium in the Erlenmeyer flask using stock solutions prepared in dimethylsulphoxide. The composition of the elicitor cocktail provides the following concentrations in the elicitation medium (+cells): 1.5 g/L sodium pyruvate, 0.440 g/L potassium pyrophosphate, 0.0004 g/L 2iP, 0.036 g/L methyl jasmonate and 2 g/L chitin.

The production of celastrol and derivatives thereof is conducted for 12 days in the dark at 27-28° C. with orbital shaking at 120 rpm.

Harvesting of the Biomass for Extraction:

When the culture stops, the medium is filtered to recover the totality of the biomass containing the majority of celastrol.

After separation of the biomass by 0-50 μm filtration, 2 volumes of ester-type solvent and more particularly alkyl acetate, notably ethyl acetate (or isopropyl acetate), are mixed with 1 weight of biomass. This mixture is extracted by sonication to lyse the cells and make the cytosol components available. The organic phase comprising the triterpenic fraction is recovered after centrifugation of the mixture.

The celastrol concentration in the organic phase is measured. The celastrol concentration per litre of suspension is estimated at 553 mg, which corresponds to a weight of 0.0166 g of celastrol per gram of dry cell weight.

### Example 4

### Production of Triterpenes (Celastrol and Derivatives) in Wave-Type Single-Use Bioreactor Bags

The example illustrated is described for wave-type reactors, for example, Sartorius brand, for volumes of 5 L or 10 L or 2×5 L, but the method can be adapted and applied to larger volumes and to equipment from other manufacturers.

The binary system previously described for traditional laboratory bioreactors made of glass or industrial bioreactors made of stainless steel is applied in the same way with the 2 wave bags.

Proliferation:

Wave bioreactor A (5 L), placed on its platform, is filled with the proliferation medium by inline sterilizing filtration and inflated with air.

A Tripterygium wilfordii pre-culture is prepared for 15 days in an Erlenmeyer flask as described in example 2. The proliferation medium of the bioreactor is then seeded with this preculture at a concentration of 160 g/L (bag A).

The bioreactor is incubated according to the following conditions:
rocking angle: 5-8°;
rocking frequency: 16-30 rpm;
aeration rate: 0.1-0.5 L/min of air enriched to 50% pure oxygen;
temperature: 27° C.;
duration: 17 days.

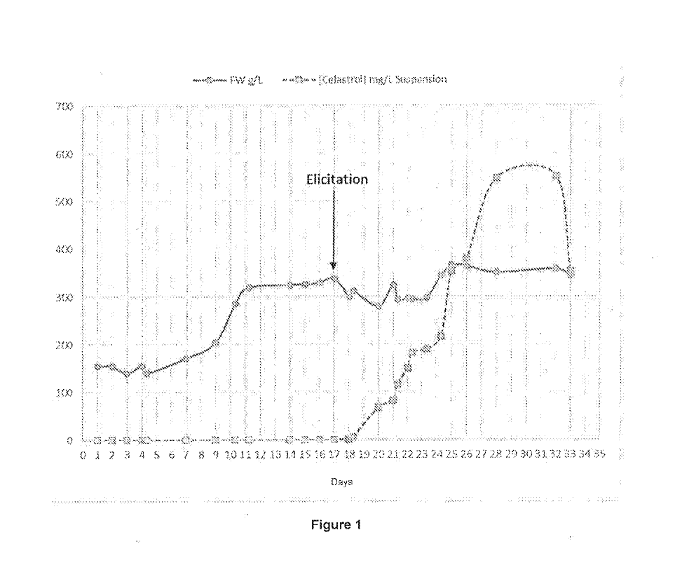

During this proliferation phase, cell growth is measured daily (FIG. 1).

Elicitation and Production of Triterpenes (Celastrol and Derivatives):

A volume of about 1000 ml of culture from bag A (5 L) is transferred to bag B placed next to bag A on the same platform. The bag A medium is supplemented with 1000 mL of concentrated medium in demineralized water having the following composition: Macroelements: NH₄NO₃ at 13.9 g/L, KNO₃ at 15.2 g/L, CaCl₂.2H₂O at 2.2 g/L, MgSO₄.7H₂O at 370 mg/L, KH₂PO₄ at 170 mg/L; Microelements: KI at 0.83 mg/L, H₃BO₃, at 31.2 mg/L, MnSO₄.4H₂O at 91.5 mg/L, ZnSO₄.H₂O at 33.05 mg/L, Na₂MoO₄.2H₂O at 1.25 mg/L, CuSO₄.5H₂O at 0.125 mg/L, CoCl₂.6H₂O at 0.125 mg/L, FeSO₄.7H₂O at 139 mg/L, Na₂EDTA.2H₂O at 186.5 mg/L; Vitamins: myo-inositol at 1250 mg/L, nicotinic acid at 8.5 mg/L, pyridoxine-HCl at 5 mg/L, thiamine-HCl at 5 mg/L; Amino acid: glycine at 20 mg/L; Carbon source: sucrose at 150 g/L; to which is added the following elicitation cocktail: 7.5 g/L sodium pyruvate, 2.2 g/L potassium pyrophosphate, 0.002 g/L 2iP, 1.8 g/L methyl jasmonate and 10 g/L chitin.

The contents of bag A are then elicited with shaking at a temperature of 27° C.

Culture in elicitation phase is followed by measurement of cell growth and celastrol concentration in the culture for 16 days (FIG. 1).

It can be seen that the celastrol concentration in bag A increases steadily up to day 32 and is at its maximum at 553 mg per litre of elicitation medium after 15 days of incubation.

The celastrol production rate is about 46 mg/L of cell suspension per day for 15 days after elicitation up to day 32.

The kinetics of celastrol production begin to shift shortly after almost all available sucrose has been consumed (FIG. 1).

### Example 5

### Production of an Extract Enriched in Celastrol by Solid/Liquid Extraction of Biomass from a Tripterygium wilfordii Cell Suspension (TW08)

At 15 days after elicitation (32 days after inoculation), most of the biomass is recovered by filtration of the cell suspension with a nylon filter (20-50 μM) (TW08).

From 5 L of suspension, about 1925 g of biomass is recovered. This biomass is extracted with ethyl acetate (or isopropyl acetate) in a proportion of 2:1 (Vol:Weight) based on the weight of biomass (here 3850 mL of solvent for 1925 g of biomass). The biomass/solvent mixture is then subjected to physical extraction, by sonication. The organic phase is then recovered after maceration with agitation. The addition of solvent (followed by maceration with agitation and recovery of the organic phase) is repeated 2 times.

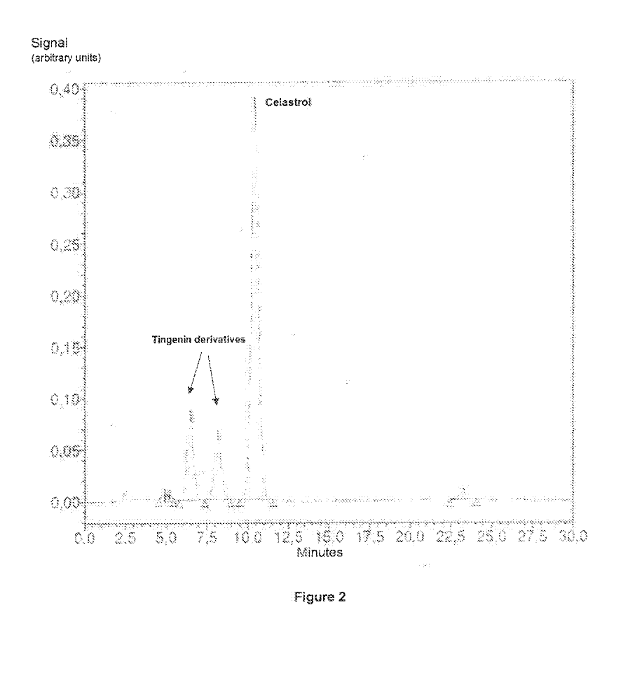

The concentration of celastrol and derivatives in the organic phase is measured by HPLC assay (quantification of celastrol, tingenin derivatives) (see FIG. 2) Experimental conditions: Waters Atlantis dC18 column 4.6×150 mm-5μ equipped with an Atlantis dC18 guard column 5 μm 4.6×20 mm Gd Column-5μ. Mobile phases: (A) Ammonium acetate, 0.1 mM pH 4.0; (B) Ammonium acetate, 0.1 mM pH 4.0 in 99.8% acetonitrile. Gradient: initial 25% (A)/75% (B)—20 min 0% (A)/100% (B)—24.5 min 0% (A)/100% (B)—25 min 25% (A)/75% (B)—30 min 25% (A)/75% (B). Flow rate: 1 mL/min. Detection at 426 nm: retention peaks (min): tingenin B (6.5), tingenin A (8.2), celastrol (10.5).

### Example 6

### Purification of Celastrol from the Tripterygium wilfordii Culture

From the extract obtained in example 5, ethyl acetate (or isopropyl acetate) is evaporated under reduced pressure to obtain a dry extract. Part of the extract thus obtained is purified first by mediumpressure liquid chromatography (MPLC) on silica (40 g, 125×25 mm, 30 μm) with a CH₂Cl₂/MeOH elution gradient (100/0 to 0/100). All fractions obtained are analysed by thin-layer chromatography (TLC—Stationary phase: 60 Å silica; Mobile phase: 70:33:3 toluene/ethyl acetate/acetic acid) and the fractions mainly containing celastrol and its tingenin derivatives (A and B) are collected. In a second step, the fraction containing the products of interest (celastrol and derivatives) is purified by high-performance liquid chromatography (HPLC) in reversed-phase mode (LiChrospher 100RP18, 250×25 mm, 5 μM) with a water/acetonitrile/0.1% formic acid linear gradient (80/20 to 0/100). The celastrol peak is collected and concentrated under reduced pressure; about 2.5 to 3 g of celastrol is obtained from 5 L of initial culture suspension. Optionally, it may be crystallized to have absolute purity.

In parallel, part of the dry extract obtained is taken up in a buffer to give extract R003034 which will be evaluated for anti-Th17 pharmacological activity.

### Example 7

### Anti-Th17 Activity on Human CD4+ Cells of the Celastrol-Enriched PCC Extract

In inflammatory dermatoses (notably in psoriasis), it is known that CD4+ T cells overexpress interleukins IL17, IL6 and IL22, as well as IFN-gamma and TNF-alpha. In the case of atopic dermatitis, IL17 is overexpressed in a certain phase of the disease. Furthermore, the Th17 pathway is strongly activated in acne. The inhibitory power of an enriched extract according to the invention on the overexpression of these molecules was tested.

Human CD4+ cells were isolated from blood mononuclear cells of 2 donors on Ficoll Paque Plus® according to the protocol recommended by the manufacturer (GE Healthcare). CD4+ lymphocytes are isolated by positive sorting using the Miltenyi Biotec kit (CD4) and an LS column and are resuspended in RPMI culture medium (Sigma-Aldrich: containing L-glutamine and 10% foetal calf serum) supplemented with 100 μg/ml streptomycin and 100 U/ml penicillin. Suspended lymphocytes are distributed in microplate wells. Various extracts or controls are then added to the wells:

1. negative control (the same volume of buffer without reagent is added);

2. extract R003034 at 0.06 mg/ml (celastrol titre 9 ng/ml) (final concentration in the well);

3. extract R003034 at 0.2 mg/ml (celastrol titre 30 ng/ml) (final concentration in the well);

4. extract R003034 at 0.6 mg/ml (celastrol titre 90 ng/ml) (final concentration in the well);

5. 2 μM dexamethasone (positive control) (final concentration in the well).

After 2 hours of incubation at room temperature, the CD4+ lymphocytes are activated at 37° C. for 20 hours with anti-CD3 and anti-CD28 antibodies at a final concentration of 300 ng/ml and 400 ng/ml, respectively. Anti-CD3 and anti-CD28 antibodies are known to induce a Th17-type response.

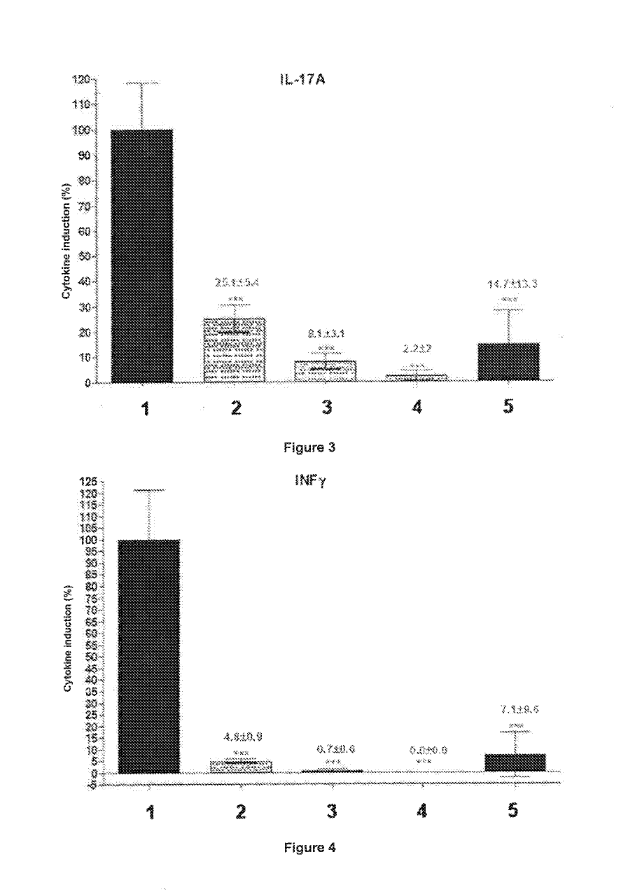

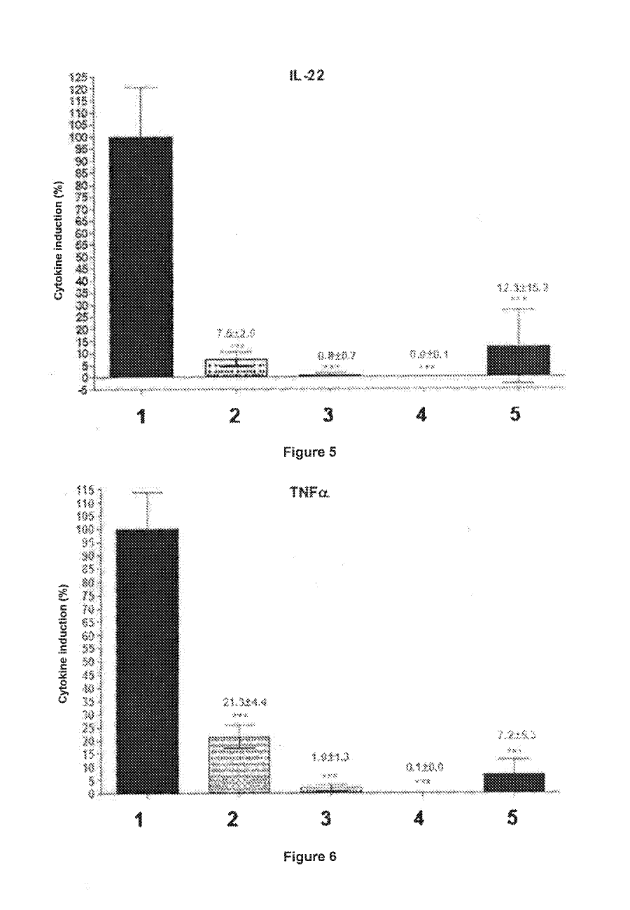

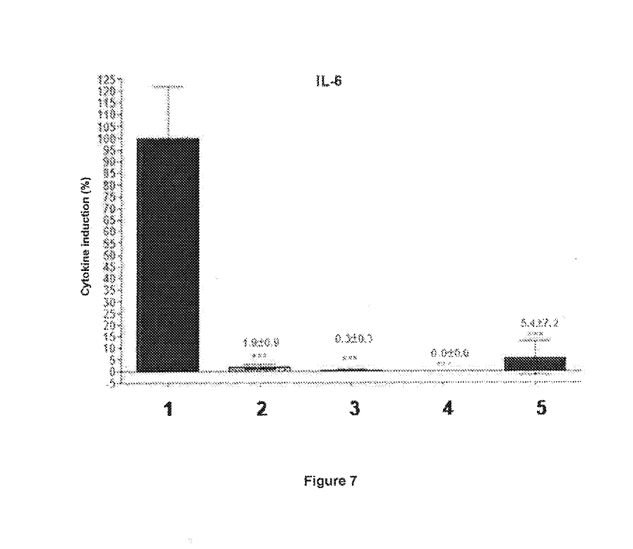

Cytokines IL-17A, INF-gamma, IL-22, TNF-alpha and IL-6 were quantified in the supernatant of each tube by the multiplex immunoassay method (Jager et at 2003). The results obtained are based on 2 experiments with lymphocytes from 2 different donors. The results are presented in FIGS. 3 to 7 as a percentage of induction compared with the negative control (1). It can be seen that:
positive control 5 (2 μM dexamethasone) inhibits the overexpression of all the cytokines: IL-17A, INF-gamma, IL-22, TNF-alpha and IL-6;
the extract according to the invention (2, 3 and 4) strongly inhibits the overexpression of cytokines IL-17A, INF-gamma, 1L-22, TNF-alpha and IL-6. The inhibition of IL-17A and TNF-alpha overexpression is dose-dependent. At the lowest dose, celastrol titre of 9 ng/ml, there is a 75% inhibition of IL-17A overexpression, an 80% inhibition of TNF-alpha overexpression, and an almost complete inhibition of INF-gamma, IL-22 and IL-6 overexpression.

This shows that extract R003034 has a very strong anti-Th17 activity, comparable or even superior to dexamethasone (2 μM), which gives it a remarkable activity in the treatment of Th17-dependent diseases such as psoriasis, acne and atopic dermatitis.

### Example 8

### Comparison of Celastrol Yields Obtained from Tripterygium wilfordii Plant Cell Cultures

Ten cultures in Erlenmeyer flasks (volume=50 mL) were carried out in parallel, from the same Tripterygium wilfordii cell line, in duplicate under the same shaking, aeration and temperature conditions. At the end of the growth phase, the density of the suspension being maximal, the amount of fresh biomass weighed was equivalent (average weight of 340 g of fresh biomass/L) in each of the Erlenmeyer flasks. At that moment, the elicitors were added to the culture medium of the ten Erlenmeyer flasks at the concentrations indicated in the table below, in which the yield is expressed in mg of celastrol/kg of fresh biomass.

| | Reference | | | | | | | | | |
|---|---|---|---|---|---|---|---|---|---|---|
| | examples | | | Examples involving the use of a cocktail of 2 elicitors | | | | | | |
| | Erlenmeyer flask | | | | | | | | | |
| | E1 | E2 | E3 | E4 | E5 | E6 | E7 | E8 | E9 | E10 |
| MeJa (μM) | 0 | 0 | 64 | 36 | 36 | 64 | 64 | 64 | 92 | 92 |
| Chitin (g/L) | 0 | 2 | 0 | 2 | 4.2 | 1.3 | 2.5 | 3.7 | 3.1 | 4.2 |
| Yield | 4.5 | 55 | 738 | 1730 | 2178 | 1506 | 1724 | 2025 | 1986 | 1878 |

The assay of suspended celastrol was performed by HPLC according to example 5, with a calibration curve prepared with control products.

The suspensions contained in Erlenmeyer flasks E1, E2, E3 were elicited with a control solution without any elicitor for E1, with chitin alone (2 g/L) for E2 and with MeJa alone (64 μM) for E3. The MeJa concentration in E3 is equivalent to that described by Liu et al. 2016, i.e. 64 μM.

The suspensions contained in Erlenmeyer flasks E4 to E10 were elicited at three different concentrations of MeJa (36, 64 and 92 μM) and five different concentrations of chitin (1.3; 2; 2.5; 3.1 and 4.2 g/L).

It is noted that the celastrol yields obtained for E6, E7 and E8 are increased by 204%, 233% and 274%, respectively, compared with E3, the MeJa concentration being the same in these four Erlenmeyer flasks. An even better yield is observed for E5, in which the MeJa concentration is different.

### Example 9

### Inhibitory Activity Against NFκB

The transcription factor NFκB controls the expression of a large number of genes involved in regulation of the inflammatory response. In the inactive state, NFκB is sequestered in the cytoplasm by the IKB protein. Certain pro-inflammatory stimuli such as TNF-alpha and IL-1-beta lead to the activation of NFκB, i.e. its nuclear translocation. Once in the nucleus, NFκB will induce the transcription of pro-inflammatory genes encoding cytokines, chemokines, adhesion molecules, growth factors and inducible enzymes.

The anti-inflammatory activities of the extracts obtained from the suspensions contained in Erlenmeyer flasks E1, E2, E3 and E6 of example 8 were evaluated in vitro on human HaCaT keratinocytes according to the method described by Albanesi et al. The extracts, weighed in dry weight, were taken up in a buffer and introduced into a HaCaT cell culture solution in equivalent final concentrations (ng/mL) for each extract and incubated for 1 hour. NFκB expression is then induced by stimulation with TNF-alpha (0.3 ng/mL).

| | | | Extract | | Extract | | Extract | | Extract | |
|---|---|---|---|---|---|---|---|---|---|---|
| | TNF- | | derived | | derived | | derived | | derived | |
| | alpha | Dex | from E1 | | from E2 | | from E3 | | from E6 | |
| Conc. | 0.3 | 2 μM | 0.19 | 0.58 | 0.19 | 0.58 | 0.19 | 0.58 | 0.19 | 0.58 |
| % inh. | 0 | 42.5 | −1 | −9 | −7 | 2 | 3 | 1 | 20 | 68 |
| sem | 4 | 6 | 2 | 3 | 3 | 3 | 2 | 3 | 3 | 4 |

Dex: Dexamethasone; Conc.: concentration (ng/mL); % inh: % inhibition of NFκB expression with respect to induction of TNF-alpha; sem: standard error of the mean.

Dexamethasone at 2 μM, used as positive control, inhibits NFκB expression by more than 40%.

The extract according to the present invention (obtained from E6, with the pair of elicitors MeJa and chitin) evaluated at concentrations of 0.19 and 0.58 ng/mL inhibits NFκB expression by 20% and 70%, respectively.

The extract prepared according to Liu et al (from E3, with MeJa as sole elicitor) evaluated at the same concentrations does not induce any appreciable inhibition of NFκB expression. Similarly, extracts derived from E2 (with chitin as sole elicitor) and from the control E1 have no effect on NFκB expression.

Only the extract derived from elicited culture according to the present invention strongly inhibits NFκB, and in a concentration-dependent manner.

### Example 10

### Antimicrobial Activity Against Propionibacterium acnes of an Extract Prepared According to the Process of the Present Invention Compared with that of Other Compounds

Activity is evaluated on Propionibacterium acnes (CIP 53117T), with an inoculation suspension at 10⁸ CFU/mL. The maintenance medium is Columbia agar+5% sheep blood, with incubation for 24 hours at 36±1° C. under anaerobic conditions. The test medium is composed of Mueller Hinton broth+10% foetal calf serum (FCS) and Columbia agar+5% sheep blood, with incubation for 24 h at 36±1° C. under anaerobic conditions. The determination of minimum inhibitory concentrations (MICs) is performed by micro-method in liquid medium. 100 μL of liquid culture medium is deposited in each well of a sterile 96-well microplate. 100 μL of the product to be tested is deposited in the first well of a row. One-half dilutions are then made from wells 1 to 10. Propionibacterium acnes test suspensions are prepared extemporaneously in tryptone salt. 100 μL is deposited in each well of a second microplate, except column 11. All of the first microplate is inoculated, using a Denley multipoint inoculator, from the second microplate. After incubation, MICs are defined as the highest dilution with no visible growth. Columns 11 and 12 serve as negative and positive growth control, respectively. Two references were tested in parallel. The determination of minimum bactericidal concentrations (MBCs) is performed by subculturing MIC microplates on agar medium using the Denley multipoint inoculator. After incubation, MBC is defined as the highest dilution with no visible growth. All tests are performed in duplicate.

The compounds tested are:

purified celastrol in DMSO (stock solution=150 μg/mL/10% DMSO)

the extract prepared according to the process of the present invention in pentylene glycol (stock solution=2%/2% pentylene glycol)

tingenin A in DMSO (stock solution=150 μg/mL/10% DMSO)

tingenin B in DMSO (stock solution=150 μg/mL/10% DMSO)

the references are amoxicillin, DMSO (stock solution=100%), and pentylene glycol (stock solution=2%)

The results obtained are summarized in the table below, which shows the MIC and MBC values for the 4 samples and both excipients. The maximum test concentration corresponds to the concentration of the stock solution/2.

| Compounds | MIC (μg/mL) | MBC (μg/mL) |
|---|---|---|
| Celastrol | 0.59 | 1.17 |
| DMSO | 5 | 5 |
| Extract according to the process | 0.25* | 0.25* |
| Pentylene glycol | >1 | >1 |
| Tingenin A | 2.34 | 2.34 |
| Tingenin B | 1.17 | 1.17 |

*or 0.375 μg/mL in celastrol equivalent.

In conclusion, celastrol has a quite attractive antibacterial activity on Propionibacterium acnes, but it appears that it is the extract prepared according to the process of the invention that has the highest level of antibacterial activity on Propionibacterium acnes. Tingenin B has higher antibacterial activity than that observed with tingenin A.

### Example 11

### Antimicrobial Activity Against Staphylococcus aureus of an Extract Prepared According to the Process of the Present Invention Compared with that of Other Compounds

It is known that S. aureus is a pathogen which colonizes the lesions of patients with atopic dermatitis and that it is also present in patients with psoriasis (Tomi et al. 2005).

The antibacterial activity against S. aureus of the extract obtained according to the process of the present invention was evaluated. The experiment was performed as described in example 10, with the pathogenic strain S. aureus CIP 4.83, the maintenance medium Trypticase Soya Agar, the test medium MIC/MBC broth and Mueller Hinton agar and incubation at 36° C. for 24 h.

The results obtained are summarized in the table below. It can be seen that while celastrol has a high antibacterial activity, the anti-S. aureus activity of the extract obtained according to the process of the present invention titrated in celastrol equivalent is even better. Furthermore, the tingenin B molecule has a better activity than tingenin A.

| Compounds | MIC (μg/mL) | MBC (μg/mL) |
|---|---|---|
| Celastrol | 0.29 | 0.59 |
| DMSO | >5 | >5 |
| Extract according to the process | 0.125* | 0.25** |
| Pentylene glycol | >1 | >1 |
| Tingenin A | 1.17-2.34 | 2.34 |
| Tingenin B | 0.59 | 1.17 |

*0.1875 μg/mL in celastrol equivalent;
**0.375 μg/mL in celastrol equivalent.

In conclusion, this extract has not only an attractive anti-Th17 pharmacological activity, but surprisingly a strong anti-S. aureus activity, which makes it very attractive as active agent in the topical treatment of atopic dermatitis and psoriasis.

### Example 12

### Cream for Topical Application

| Ingredients | Mass percentage |
|---|---|
| glycerine | 8 to 10% |
| hydroxyacrylate copolymer | 1.8 to 2% |
| xanthan gum | 0.1 to 0.3% |
| ceteareth 33 and cetearyl alcohol | 4 to 6% |
| glyceryl stearate | 1.5 to 2% |
| ethylhexyl palmitate | 12 to 15% |
| Tripterygium wilfordii extract (in | 0.1 to 2.0% |
| solution; 10 to 600 mg/L) | |
| glycolic acid | 2 to 4% |
| Lactamide MEA | 3 to 6% |
| Laureth-9 | 1 to 3% |
| sodium shale oil sulphonate | 1 to 3% |
| chicory root extract | 1 to 3% |
| Water made up to | 100% |

The Tripterygium wilfordii extract is prepared according to the present invention, its concentration being expressed as weight of dry extract in a solvent compatible with the formulation, such as olive oil, pentylene glycol or myritol 318 or others.

This exemplary formulation is by no means limiting and may be adapted according to the treatment.

Thus, for treatment of the scalp, a shampoo may be formulated by calling upon on the knowledge of the person skilled in the art.

### REFERENCES

Camelio et al. JACS 2015, 137; 11864-67
Coppede et al. Plant Cell Tiss. Organ Cult. 2014, 118:33-43
Jager et al. Clin. Diagn. Lab. Immunol. 2003, 10(1):133-9
Kelhala et al. PLOS One 2014, 9(8):e105238
Liu et al. J. Asian Nat. Prod. Res. 2016, 19:1-10
Lowes et al. Annu. Rev. Immunol. 2014, 32:227
Miyagaki et al. J. Derm. Science 2015, 78:89
Murashige & Skoog Physiol. 1962, 15: 473-497
Albanesi et al. Curr. Drug Targets Inflamm. Allergy 2005, 4(3):329-334
Tomi et al. J. Am. Acad. Dermatol. 2005, 53(1):67-72.

1. A process for producing a crude extract enriched in pentacyclic triterpenes comprising the following steps:
(i) a phase of proliferation of cells of a plant of the Celastraceae family in a proliferation medium,
(ii) a phase of elicitation by adding an elicitation cocktail to the cell culture obtained in step (i), said elicitation cocktail comprising at least one monocarboxylic compound-type elicitor and at least one biotic elicitor, said biotic elicitor being an N-acetylaminoglucosamine, and
(iii) preparation of a crude extract enriched in pentacyclic triterpenes from the cell culture obtained in step (ii).

2. The process for producing an enriched crude extract according to claim 1, wherein the biotic elicitor is chitin.

3. The process for producing an enriched crude extract according to claim 1, wherein the monocarboxylic compound-type elicitor is a methyl ester.

4. The process for producing an enriched crude extract according to claim 1, wherein the at least one monocarboxylic compound-type elicitor is methyl jasmonate and the at least one biotic elicitor is chitin.

5. The process for producing an enriched crude extract according to claim 1, wherein the elicitation cocktail further comprises at least one cell differentiation factor for plant cells and at least one precursor of the terpene synthesis pathway.

6. The process for producing an enriched crude extract according to claim 5, wherein the at least one cell differentiation factor for plant cells is selected from a cytokine, a gibberellin and a mixture thereof.

7. The process for producing an enriched crude extract according to claim 5, wherein the at least one precursor of terpene synthesis is selected from the group consisting of sodium pyruvate, potassium pyrophosphate and a mixture thereof.

8. The process for producing an enriched crude extract according to claim 1, wherein the pentacyclic triterpene is selected from the group consisting of celastrol, tingenin A, tingenin B, pristimerin, tripterygone and mixtures thereof.

9. The process for producing an enriched crude extract according to claim 8, wherein the pentacyclic triterpene is celastrol.

10. The process for producing an enriched crude extract according to claim 1, wherein:
(i) the plant of the Celastraceae family is Tripterygium wilfordii,
(ii) the at least one monocarboxylic compound-type elicitor is methyl jasmonate and the at least one biotic elicitor is chitin, and
(iii) the pentacyclic triterpene is celastrol.

11. The process for producing an enriched crude extract according to claim 1, further comprising a step (iv) of obtaining a purified extract of one or more pentacyclic triterpenes from the enriched crude extract obtained in step (iii).

12. An enriched crude extract obtainable by the process according to claim 1.

13-15. (canceled)

16. A pharmaceutical or dermocosmetic composition comprising an enriched crude extract according to claim 12.

17. The process for producing an enriched crude extract according to claim 3, wherein the monocarboxylic compound-type elicitor is methyl jasmonate.

18. The process for producing an enriched crude extract according to claim 6, wherein the at least one cell differentiation factor for plant cells is selected from benzylaminopurine (BAP), 6-γ,γ-dimethylallylaminopurine (2iP) and a mixture thereof.

19. A method for treating an inflammatory dermatosis inducing a TH17-type immune response comprising the administration to a person in need thereof of an effective amount of an enriched crude extract according to claim 12.

20. The method according to claim 19, wherein the inflammatory dermatosis is selected from the group consisting of psoriasis, atopic dermatitis and acne.
